(19) United States
(12) Patent Application Publication
(10) Pub. No.: US 2004/0068195 A1
(43) Pub. Date: Apr. 8, 2004

(54) Method and apparatus for wearable digital wireless ECG monitoring

(76) Inventors: Louis Massicotte, Quebec (CA); Jean-Francois Montplaisir, Quebec (CA); Eric Blondeau, Ste-Foy (CA)

Correspondence Address: OGILVY RENAULT, 1981 MCGILL COLLEGE AVENUE, SUITE 1600, MONTREAL, QC, H3A2Y3, CA

(21) Appl. No.: 10/262,662

(22) Filed: Oct. 2, 2002

(51) Int. Cl. A61B 5/0402

(52) U.S. Cl. 600/509

(57) ABSTRACT

The present invention is a wearable digital wireless ECG monitoring system. The device is made of two dependant parts: the wireless digital ECG and the wireless central device. The wireless digital ECG is preferably worn on the chest. It is made of two electrodes and one data processing and transmission module. The data is collected as a full ECG curve and is digitalized inside the electrodes. After being digitalized, the signal is sent wirelessly to the central module.

### FIELD OF THE INVENTION

The invention relates to monitoring heart activity and in particular to wearable or portable electrocardiogram monitors.

### BACKGROUND OF THE INVENTION

Heart diseases are increasingly common in adults of all ages. Recent statistics have stated that sixty million North Americans suffer from heart disease. Because the North American society is getting older, the risk of suffering from heart diseases increases every year. People are now more aware of their health and need ways to apply preventive medicine.

An electrocardiogram (ECG/EKG) is an electrical recording of the heart that is used in the investigation of heart disease. Cardiologists have confirmed the urgent need for devices that can be worn for a long period to provide an ECG covering more than twenty-four hours. The idea is to enable the observation of cardiac events that are not regularly present in heart activity.

Cardiac contractions are the result of a well orchestrated electrical phenomenon called depolarization. Cell membranes move from their negative resting potential to a more positive threshold which ultimately stimulates them to contract. In the myocardium there are specialized fibers that are very conductive and allow the rapid transmission of electrical impulses across the muscle, telling them to contract. In order to maximize the force of the contraction there is uniformity in the sequence. That is, the atria contract, then the ventricles contract. This allows both sets to fill properly before ejecting the blood to its next destination. These two sections are independent, yet linked to a single impulse, (in a healthy heart,) initiated by the sinoatrial, (or sinus) node. The tissue around the valves helps to channel the impulse from the sinus node through another collection of specialized tissue, the atrioventricular node, that is situated between the two sets of chambers. This area allows slightly slower transmission of the impulse to the ventricles, allowing the atria to empty into the ventricles before they contract and force the blood to the lungs or body. This area, the AN Node, slows the impulse down to about one twenty-fifth of the original signal then passes it through to the atrioventricular bundle, or the bundle of His. This bundle divides itself into two distinct tracts through the ventricles, the bundle branches, and on to the Purkinje fibers, where the muscle of the ventricle is stimulated to contract from the bottom up, maximizing the force of ejection.

An electrical current in the direction towards the positive end of a bipolar electrode causes a positive deflection of the stylus of the ECG. If the number of myocardial cells (dipoles) in this direction increases, the current will increase as well. The greater the current, the more positive the voltage. An electrical current in the direction away from the positive end of a bipolar electrode causes a negative deflection of the stylus of the ECG. If the number of myocardial cells (dipoles) in this direction increases, the current will increase as well. The greater the current, the more negative the voltage.

The ECG Library authored by Dean Jenkins and Stephen Gerred and found on the Internet at http://www.ecglibrary.com/ in September 2002 is a very good source of information on ECGs.

An article of particular interest with respect to artificial intelligence in medical devices was published by Ralph Begley et al. in March 2000 in the Medical Device & Diagnostic Industry Magazine at page 150 and is entitled “Adding Intelligence to Medical Devices”. This article can be found on the Internet in September 2002 at the Medical Devicelink Site at http://www.devicelink.com/mddi/archive/00/03/014.html.

Most prior art systems are not powerful enough or portable enough to be worn over long periods of time. Standard Holter monitors are expensive, bulky and solely record the ECG without further analysis.

Most portable ECGs currently available on watches or the like can only record heartbeat. Although this is sufficient to determine if a patient is under cardiac arrest, it is insufficient to detect other cardiac anomalies, defects and diseases.

Prior art portable monitor systems are manufactured by a few companies, such as the Biolog™ portable ECG by Lyppard, the CCW-CAS Cardio Perfect CE™ resting ECG system by Cardio Control, the PocketView™ 12 Lead portable ECG system by Numed, the Portable ECG/Respiration Monitor by Harvard Apparatus and the Digital Angel™ Safety and Location Monitor, ThermAlert™ Watch and Alerts by Digital Angel™Corporation. These monitoring devices allow partial collection of the patient's ECG data but do not offer full collection and analysis of the data, detection of anomalies and transmission of alarms and integration with traditional medical equipment and emergency central stations. Because of these drawbacks, they cannot be used to replace traditional Holter readings and cannot ensure the patient's safety.

### SUMMARY OF THE INVENTION

Accordingly, an object of the present invention is to provide a full ECG monitor that can be worn at any time of the day and can tolerate a level of muscle activity and still be able to record normal heart activity.

Yet another object of the present invention is to reduce the impact of wire movement of the electrodes while improving signal quality for the ECG data.

Still another object of the present invention is to improve the ergonomics of the device to render the wearing of the device more pleasant to the patient.

The present invention is a wearable digital wireless ECG monitoring system. The device is made of two dependant parts: the wireless digital ECG and the wireless central device. The wireless digital ECG is preferably worn on the chest. It is made of two electrodes and one data processing and transmission module. The data is collected as a full ECG curve and is digitalized inside the electrodes. After being digitalized, the signal is sent wirelessly to a central module for analysis.

According to one broad aspect of the present invention, there is provided an electrocardiogram monitoring system for a patient having a heart and for which a heart signal is to be monitored. The system comprises a first electrode adhered to a first portion of skin of the patient; a second electrode adhered to a second portion of skin of the patient; a data acquisition unit for receiving and storing a differential signal from the first and second electrodes, the data acquisition unit being electrically connected to the first and second electrodes; and an attachment connecting the data acquisition unit to at least one of the first and second electrodes; wherein the data acquisition unit is fully supported by at least one of the first and second electrodes and is positioned close to at least one of the first and second electrodes; whereby the electrocardiogram monitoring system can be worn by the patient throughout normal day-to-day activities without disturbing the patient because there are no long wires between the electrodes and the data acquisition unit.

According to another broad aspect of the present invention, there is provided a method for monitoring a heart signal for a patient having a heart. The method comprises providing a first electrode and adhering the first electrode to a first portion of skin of the patient; providing a second electrode and adhering the second electrode to a second portion of skin of the patient; receiving and storing in a data acquisition unit a differential signal from the first and second electrodes; and attaching the data acquisition unit to at least one of the first and second electrodes; wherein the data acquisition unit is fully supported by at least one of the first and second electrodes and is positioned close to at least one of the first and second electrodes; whereby the electrocardiogram monitoring system can be worn by the patient throughout normal day-to-day activities without disturbing the patient because there are no long wires between the electrodes and the data acquisition unit.

### BRIEF DESCRIPTION OF THE DRAWINGS

These and other features, aspects and advantages of the present invention will become better understood with regard to the following description and accompanying drawings wherein:

FIG. 1 is block diagram of the main components of the preferred embodiment;

FIG. 2 is a graphical representation of the wearable device of the preferred embodiment;

FIG. 3 is a graphical representation of the user interface of the computer;

FIG. 4 is a graphical representation of an ECG wave;

FIG. 5A and FIG. 5B are block diagrams of the electrodes;

FIG. 6 is a detail of a 3M Red Dot™ electrode;

FIG. 7 is a block diagram of the transformation of the electrode signal into a wireless output;

FIGS. 8A and 8B are, respectively, top and bottom views of a realization of the left part electrode;

FIGS. 9A and 9B are, respectively, top and bottom views of a realization of the right part electrode;

FIG. 10A and 10B are, respectively, top and bottom view of a realization of the electrode signal processing module;

FIG. 11 is a block diagram of the components of the emergency transmitter module; and

FIGS. 12A and 12B are, respectively, top and bottom views of a realization of the central unit.

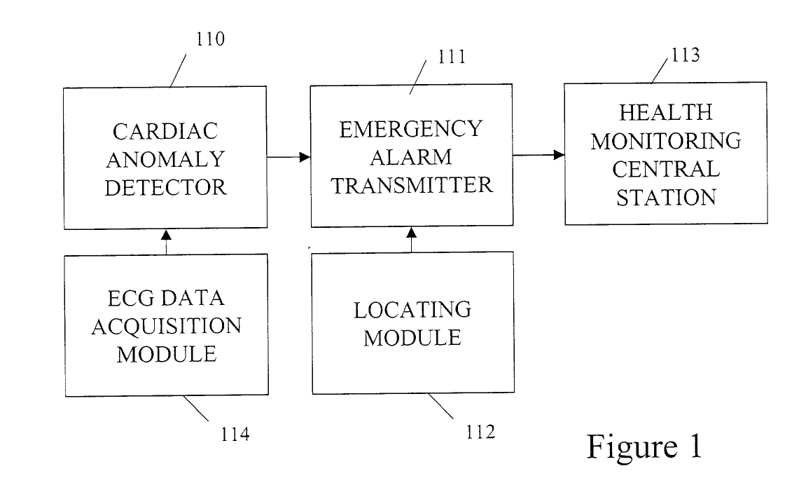

### DETAILED DESCRIPTION OF THE PREFERRED EMBODIMENT

While illustrated in the block diagrams as groups of discrete components communicating with each other via distinct data signal connections, it will be understood by those skilled in the art that the preferred embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present preferred embodiment.

The present invention is for a full ECG monitoring device 114 which collects the ECG data from the patient and transmits it.

The ECG data acquisition module 114 is preferably a full wireless ECG system which ensures that the patient can attend his day-to-day activities without being held back by the wires of the electrodes. Movements of the wires in a standard ECG system typically create interference in the data collected by the electrodes. This interference is greatly reduced by the present invention.

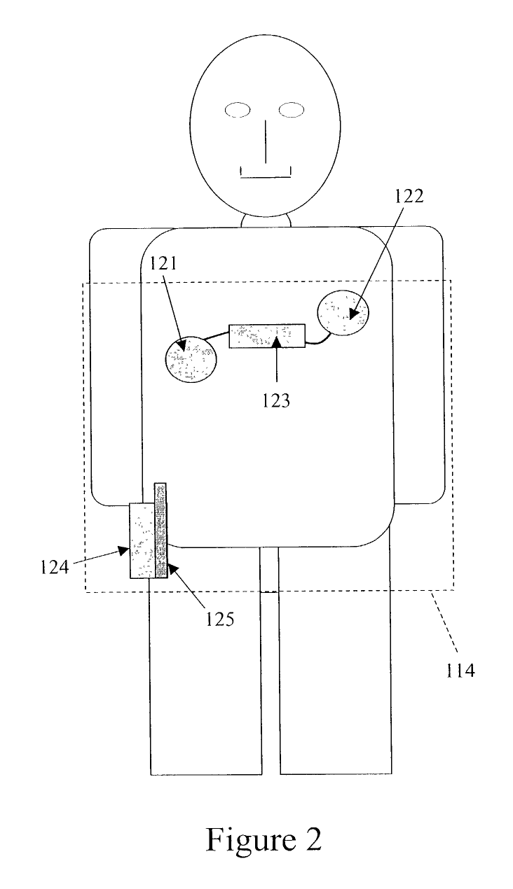

With reference to FIG. 2, the ECG data acquisition module 114 preferably comprises two electrodes 121 and 122 which are applied to the body of the patient and which perform data acquisition to produce a differential signal. The acquired data is then processed in an electrode signal processor 123 which performs digital sampling and digital modulation and sends the acquired data on Radio Frequency (RF). The digital sampling is done to reduce noise from interferences and magnetic fields. The distance traveled by the low voltage of the heart to the electrodes is reduced thereby creating a more precise curve of the heart activity.

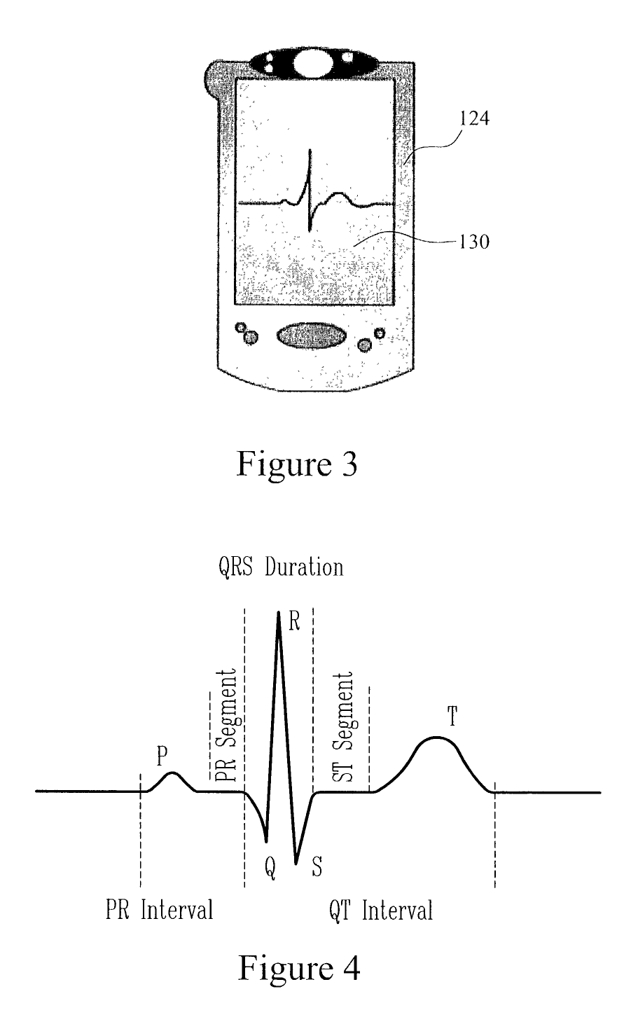

The ECG system is preferably used in conjunction with a data receiver 125 which is a wireless portable device which can be worn on the patient's belt, in his pocket or even in a bag that he is carrying. The data receiver 125 can be connected to a computer 124, a hand-held PC, a PALM™ Pilot, a cellular or any other device which is compatible with the RS-232 protocol. The acquired data can then be displayed (see FIG. 3) on a small matrix screen of the data receiver 125 and/or on the screen 130 of the computer 124. The data acquired is typically of the type shown in FIG. 4. A plurality of filters are used on the acquired data to enhance the clarity of the ECG curve obtained and to extract precise information on the patient's heart. This is fully described in Applicants' co-pending U.S. patent application Ser. No. ______ filed simultaneously on Oct. ______, (attorney docket number 15063-3us) the specification of which is hereby incorporated by reference.

The preferred embodiment of the ECG data acquisition module 114 is divided in two parts: the first portion, the electrodes and the electrode signal processor and transmitter 121, 122, 123 capture and convert the heart signal and send it wirelessly in digital form to a second component, which is optional, the electrode signal receiver 125 that receives the signal via a RS-232 port and can communicate with a computer 124.

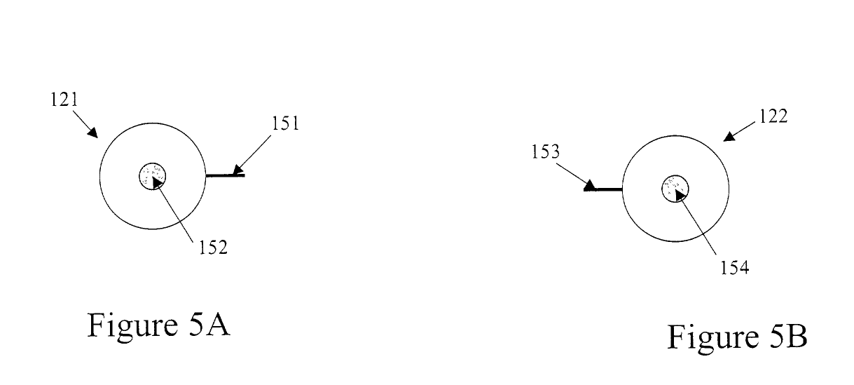

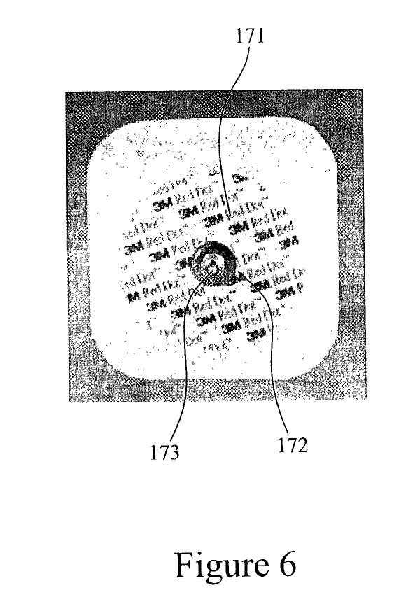

Referring now to FIGS. 5A and 5B, sockets 152 and 154 of electrodes 121 and 122 are preferably each connected to a RedDot™ diaphoretic monitoring electrode manufactured by 3M (see FIG. 6). This electrode is commonly used in hospitals. Each electrode has two functions: first to intercept the electrical signal produced by the heart and second to attach the electrode to the patient's body. To ensure an adequate signal, the right electrode 121 is preferably placed beneath the right breast and the left electrode 122 is preferably placed above the left breast as is shown in FIG. 2. Wires 151 and 153 are used to connect the electrodes 121, 122 to the electrode signal processor 123.

The attachment of the electrode signal processor 123 to the electrodes can be done in various ways. Preferably, the electrode signal processor 123 is mounted directly on one of the electrodes while being electrically connected to the other electrode. In another embodiment, as is shown in FIG. 2, the electrode signal processor 123 is hung between the two electrodes using wires which attach to the sockets 152 and 154. Because the electrode signal processor 123 is manufactured to be as small as possible, it is possible to hang the electrode signal processor 123 halfway between the electrodes 121 and 122 so that the electrode signal processor 123 may lie on the patient's chest.

FIG. 6 is a detail of a 3M Red Dot™ electrode. It comprises an adhesive portion 171 to contact the skin of the patient directly, a metallic electrode 172 to read the voltage signal from the heart and a socket 173 for attachment to other modules and to connect wires.

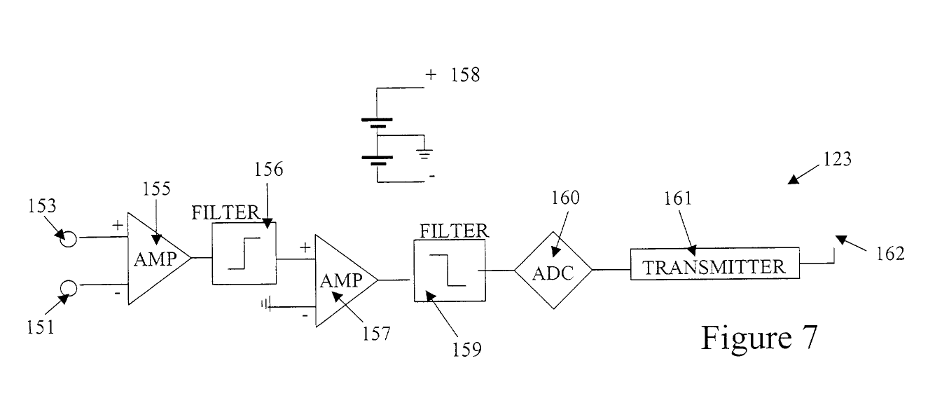

FIG. 7 shows the steps needed to produce a wireless output of the electrodes signal. The output of the electrodes is connected via wires 151 and 153 to the inputs of the electrode signal processor 123. The right electrode is connected to the ground and to the reference pin of the amplifier and the left electrode is connected to the negative input of the amplifier. The differential signal then goes through a low-power instrumentation amplifier 155. This instrumentation amplifier provides good high gain and low noise amplification of the electrode differential signal. This amplifier eliminates the noise signal produced by the line sector. The noise commonly produced by the line sector (60 Hz) that interferes with the ECG signal (0.5 Hz to 150 Hz) is reduced by the fact that this noise appears on the positive and the negative inputs of the instrumentation amplifier. So the difference between the two inputs subtracts the noise from the ECG signal. The voltage difference between the two electrodes is filtered to a high pass filter 156 with a cut frequency of 0.5 Hz. This filter also eliminates the DC signal present on the ECG reading.

A second amplification 157 of the signal provides a total amplification ratio of 1000 (1 v/1 mv), improving the ratio between the heart signal and the noise signal. Then the heart signal is fed to a low pass filter 159 to eliminate frequencies above 150 Hz. The output signal produced by the two amplifiers and filtered between 0.5 Hz and 150 Hz is fed to an analog-to-digital converter 160 which outputs an 8-bit serial signal. The format of the signal is RS-232 compatible. The signal is then modulated to a digital FM transmitter 161. The output signal of the transmitter is fed to an antenna 162 for RF radiation. The entire circuit is powered by batteries 158 which produce a power feed between −3 volts and 5 volts.

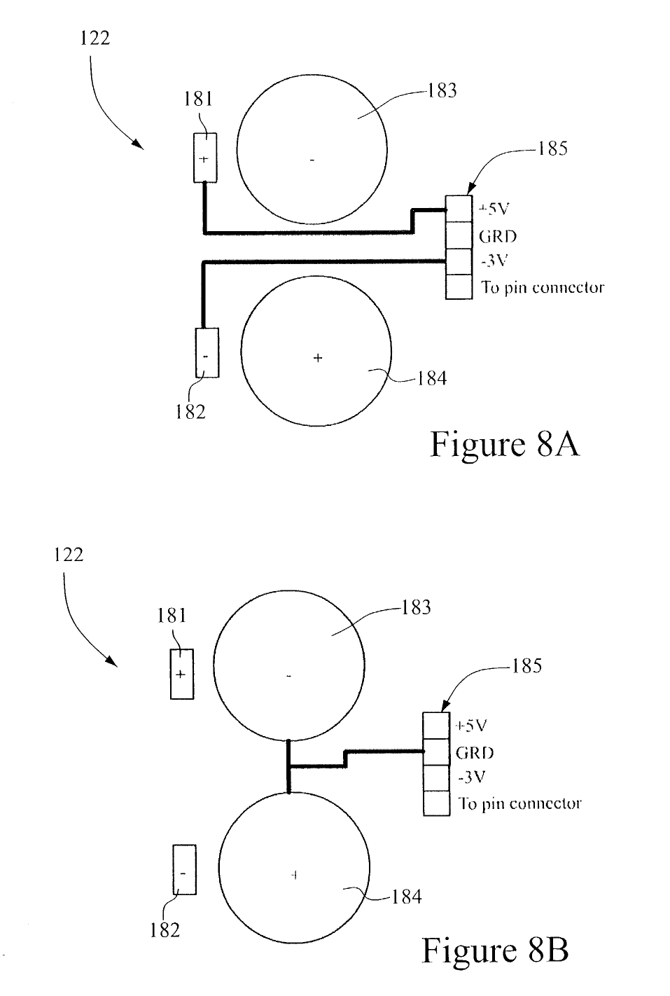

FIGS. 8A and 8B are, respectively, top and bottom views of a realization of the left part electrode 122. The left part electrode 122 comprises a lithium batteries socket where the negative input 183 is connected to the ground and to a positive input 181 to provide the 5V of the ECG. It further comprises a lithium batteries socket where the positive input 184 is connected to the ground and to a negative input 182 to provide the −3V of the ECG. Outputs 185 are as follows: a ground connection, a socket for the pin connector, a −3V output and a +5V output.

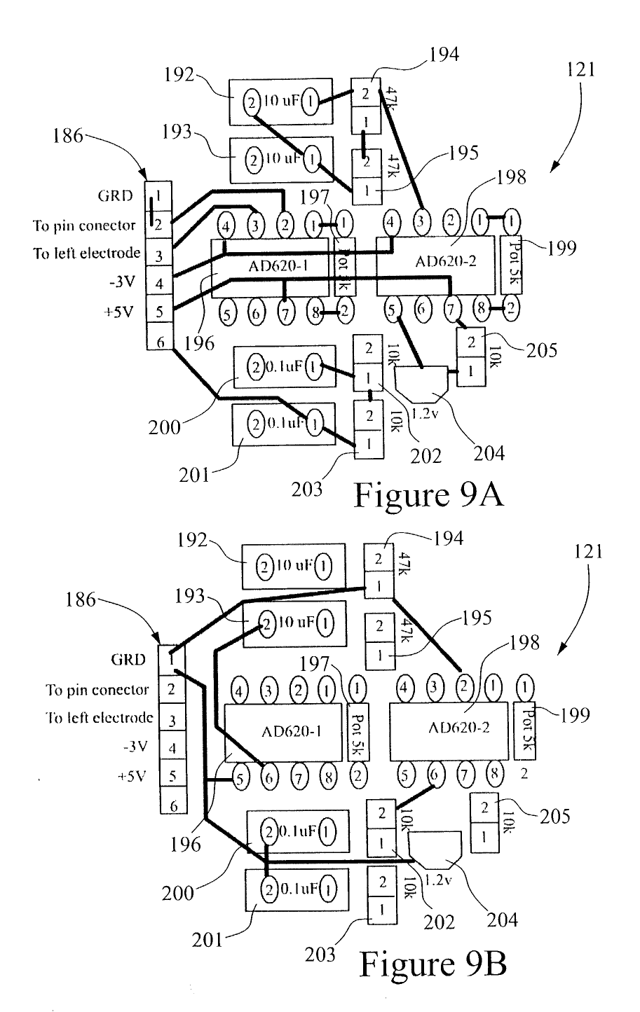

FIGS. 9A and 9B are, respectively, top and bottom views of a realization of the right part electrode 121. In the present embodiment, the right part electrode 121 comprises an amplification circuitry and a first portion of the signal processing. It would be possible to realize the invention by implementing the electronics into the left part electrode and having a right part electrode similar to that of FIGS. 8A and 8B. The electrode 121 preferably comprises two AD620AN 196 and 198, two 10 uF capacitors 192 and 193, two 0.1 uF capacitors 200 and 201, two 47K resistors 194 and 195, three 10K Resistors 202, 203 and 205, two 5K Potentiometers 197 and 199 and a 1.2V LM385BZ voltage regulator 204. Inputs and Outputs 186 are as follows: a ground connection, a socket for the pin connector (left electrode), a socket for the right electrode, a −3V input and a +5V input. Pin 6 of inputs and outputs 186 is the output of the ECG signal. The preferred dimensions for the Left and Right electrodes are 2 cm×2 cm×1 cm.

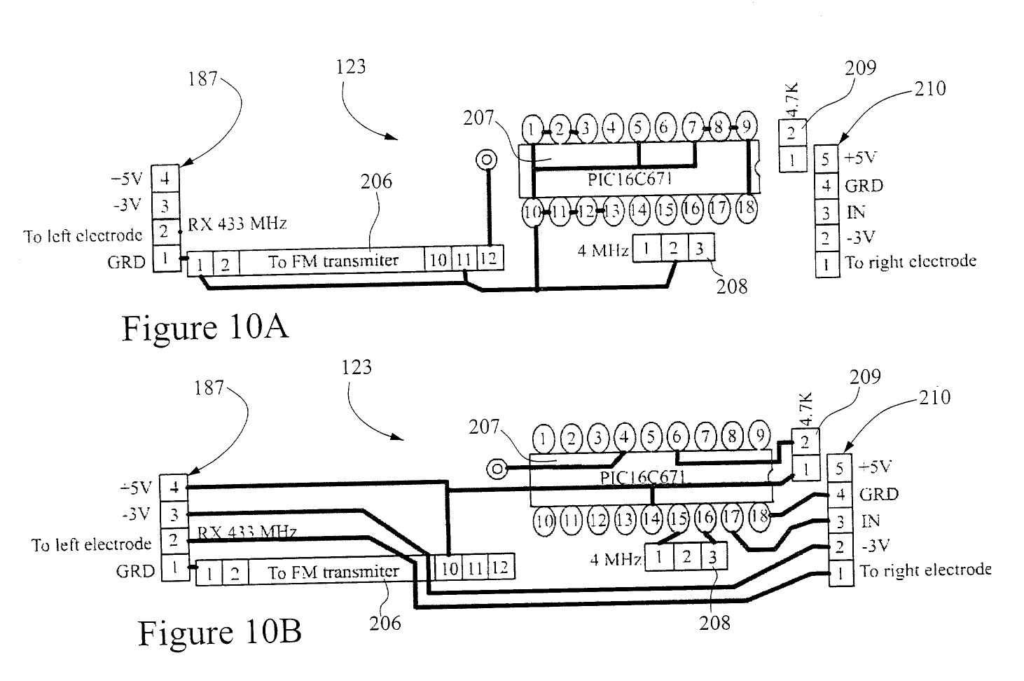

FIGS. 10A and 10B are, respectively, top and bottom view of a realization of the electrode signal processor 123 linked to the two electrodes, having a transmitter and comprising a 4 MHz PIC 16C671 207 Micro controller, a 4 MHz resonator 208, a 4.7K resistor 209 and a FM transmitter TXM-433 206. It also has proper connections 187 and 210 to the wires coming from the electrodes. The preferred dimensions for the electrode signal processor are 6 cm X 1.5 cm X 1 cm.

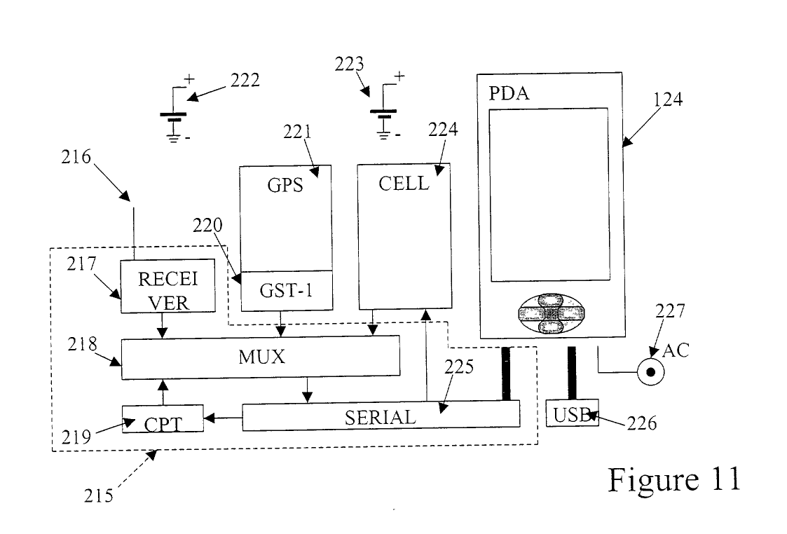

Additionally, the signal from the transmitter antenna 162 can intercepted by the receiver antenna 216 of the central unit 215 as shown in FIG. 11, and fed to a digital FM receiver 217. This is fully described in Applicants' co-pending U.S. patent application Ser. No. ______ filed simultaneously on Oct. ______, (attorney docket number 15063-3us) the specification of which is hereby incorporated by reference.

This receiver 217 exactly reproduces the signal from the converter 160. The RS-232 compatible signal passes through a 4:1 multiplexing device 218. The purpose of this stage is to multiplex other serial devices such as the GPS module 220, the GST-1 module 221 and the cellular phone module 224 on the same port. Device selection is made via the RS-232 RTS pin. Each state change of the RTS line acts like a clock for the counters 219 and the value of these counters results in a RS-232 line selector. When the proper line selector is set, the receiver outputs the digital signal via the serial port 225. This signal can be processed by software via a PC, Portable PC or handheld PC 124, for example, an IPAQ™ by Compaq. The computer 124 preferably has a USB port 226 and an AC power supply 227. Power sources 222 and 223 are provided in the central unit. The voltages of these power supplies depend on the type of device used in conjunction with the invention. They are typically 3 or 5 V. For the IPAQ, a 5V supply is used. The USB port 226 is used for synchronization of the portable computer 124. The AC power supply 227 is used to charge the module and the portable computer 124.

The Multiplexer module 218 is a grouping of microcontrollers and multiplexers allowing the relay between the various modules of the system. It acts in a dependent way to a principal controller who is, preferably, the portable computer module 124. It allows the simple port communication of several sources which would normally require several ports of communication. The request via lines of orders allows to access the various modules necessary to the integration of the system. It is independent of the bandwidth of the various components.

The preferred locating module 112 is a GPS module 221 manufactured by DeLorme according to Rockwell standards. To simplify the translation of the Rockwell signals, a GST-1 module 220 by Byosystems is added allowing to seize a signal encrypted using Rockwell 9600 bps and to obtain a standard NMEA format at 4800 bps.

The Cellular Module 224 comprises a cellular modem module GPRS/CDMA/GSM from Motorola. The preferred connection is 14.4 kbps. The addition of the multiplexing module 218 allows the connection and the conservation of this connection even if the cellular is not the object chosen by the multiplexer. Therefore, there is a ghost opening of the port of the cellular 224 even if one does not want to listen to the cellular.

The Portable Computer Module 124 is optional. It allows to access and consult the data collected. The preferred modules are Ipaq™ by Compaq and Palm™ VII by 3Com.

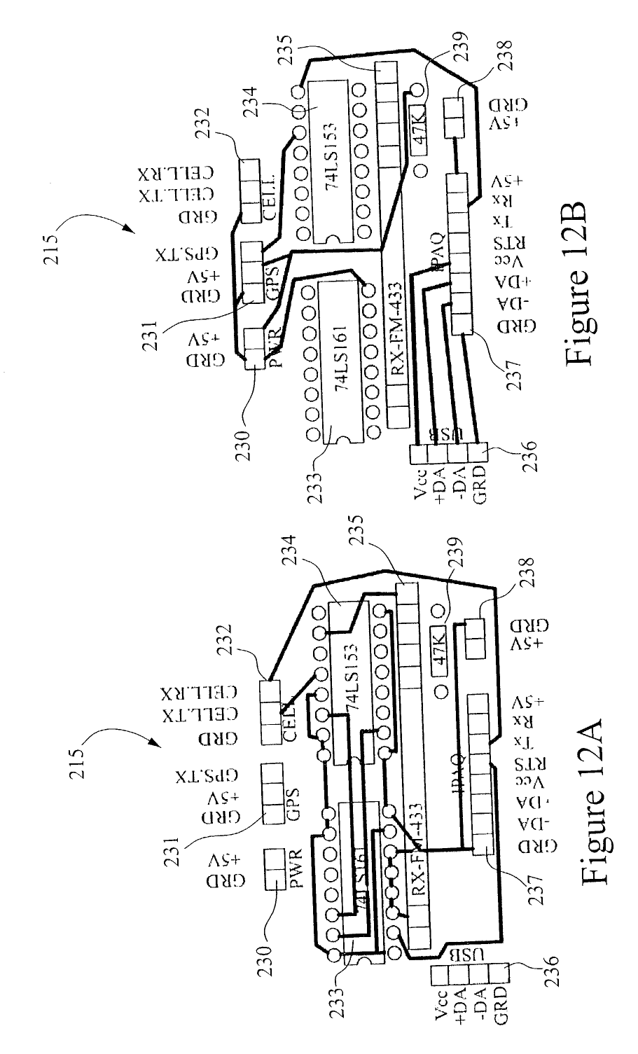

The design of the central unit 215 of FIG. 11 preferably comprises the following parts as shown in FIGS. 12A and 12B: a SILRX-433-F FM receiver 235, a 74LS153 Multiplexer 234, a Four bits synchronous 74LS161 counter 233, a 47K resistor 239 and connections to an IPAQ™ handheld computer 237, to a Motorola GPRS cellular board 232, to a DeLorme Earthmate GPS 231 and to a Bionics Rockwell GST-1 translator. The Bionics Rockwell GST-1 translator is connected directly to the DeLorme Earthmate. Connections to the USB 236 and to the power supply 238 and 230 are also provided.

At any time, the portable computer module 124 can question the multiplexer module 218 to obtain the cardiac data from the receiver 217 and the GPS data from the GPS module 221. The software analysis and the data storage are made in real time. The software does data compression based on diagrams of repetitions. At the time a cardiac event is detected, the software in the computer 124 triggers the call 111 to the digital emergency station 113 via the various modules.

At any point, the stored data can be sent to a central monitoring station for review using the emergency alarm transmitter 111. The locating module 112, which automatically takes the GPS positioning 221 of the patient every minute, tries to obtain the position again. If the last position is accurate, the system uses that location. If not, the positions of the patient in the last 10 minutes are retrieved to determine the person's movement or speed. With this data, a call is made to a central number by the emergency alarm transmitter 111 using the cellular module 224. The personal ID of the person and an ECG monitor reading of his heart activity from the ECG data acquisition module 114 are sent. This alarm message is received by the health monitoring central station 113 and the person or computer in the central station can ask for further ECG data, for example for the last hour's ECG. The entire emergency call takes less than 6 seconds and is preferably fully automated, from the trigger of the call to the forwarding of any additional ECG or anomaly data required. A person having a heart attack only has four to eight minutes to obtain medical assistance. Most of the time, a person having a heart attack is unable to dial 911 or ask for assistance himself. That is why the automated call for help is very advantageous.

The personal information given by the device to the central station is preferably the name of the patient, his medical state and history, and the ECG signal and/or trend data. As soon as the location is found, this information is also transmitted to the Emergency Alarm Station.

Thereafter, once these data are sent, connection is established between ECG module and the cellular module to create a mini-center of telemedicine in order to be able to obtain the ECG curve of the remote patient. The whole process is carried out automatically.

The Health Monitoring Central Station 113 is an Emergency Station which, contrary to a typical 911 Emergency Station, does not require a voice call to obtain the person's status and location. It is a completely digitally-enabled station which allows a emergency clerk to talk to the patient through the speakers of the handheld device he is carrying but which does not require a response from the patient to send appropriate medical assistance to the exact position of the patient. The Station is able to receive the ECG signal and follow the state of the patient. It can then relay that information to the medical team who is assigned to the patient.

The digital emergency station 113 allows the reception and remote analysis of data received by the Cardiac data acquisition module. Be it directly by modem or via Internet, the system is able to physically locate the person on a map and to thus provide to the various technicians at the Station, the data necessary to find the person as well as a constant status report. Then, it is possible to follow the status of the person by telemetry throughout the search for the person or to communicate with her or the people around her via the cellular module provided with a loudspeaker and a hands free microphone. The whole process is made automatically and requires only a few seconds in total. A station can treat more than one request at the same time.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

What is claimed is:

1. An electrocardiogram monitoring system for a patient having a heart and for which a heart signal is to be monitored, comprising:
a first electrode adhered to a first portion of skin of said patient;
a second electrode adhered to a second portion of skin of said patient;
a data acquisition unit for receiving and storing a differential signal from said first and second electrodes, said data acquisition unit being electrically connected to said first and second electrodes; and
an attachment connecting said data acquisition unit to at least one of said first and second electrodes;
wherein said data acquisition unit is fully supported by at least one of said first and second electrodes and is positioned close to at least one of said first and second electrodes;
whereby said electrocardiogram monitoring system can be worn by said patient throughout normal day-to-day activities without disturbing the patient because there are no long wires between the electrodes and the data acquisition unit.

2. An electrocardiogram monitoring system as claimed in claim 1, wherein said data acquisition unit comprises a transmitter for transmitting said differential signal to a receiver unit.

3. An electrocardiogram monitoring system as claimed in claim 1, wherein said differential signal is an analog signal.

4. An electrocardiogram monitoring system as claimed in claim 3, wherein data acquisition unit comprises an analog-to-digital converter to convert said differential signal to a digital signal.

5. An electrocardiogram monitoring system as claimed in claim 4, wherein said data acquisition unit comprises a transmitter for transmitting said digital signal to a receiver unit.

6. An electrocardiogram monitoring system as claimed in claim 1, wherein said data acquisition unit is mounted to said first electrode using said attachment and a wire is used to electrically connect said data acquisition unit to said second electrode.

7. An electrocardiogram monitoring system as claimed in claim 1, wherein said data acquisition unit is positioned half way between said first electrode and said second electrode.

8. An electrocardiogram monitoring system as claimed in claim 2, wherein said transmitter is a RF transmitter.

9. An electrocardiogram monitoring system as claimed in claim 1, wherein said first portion of skin is on a left side of said heart.

10. An electrocardiogram monitoring system as claimed in claim 1, wherein said first portion of skin is above said heart.

11. A method for monitoring a heart signal for a patient having a heart, comprising:
providing a first electrode and adhering said first electrode to a first portion of skin of said patient;
providing a second electrode and adhering said second electrode to a second portion of skin of said patient;
receiving and storing in a data acquisition unit a differential signal from said first and second electrodes; and
attaching said data acquisition unit to at least one of said first and second electrodes;
wherein said data acquisition unit is fully supported by at least one of said first and second electrodes and is positioned close to at least one of said first and second electrodes;
whereby said electrocardiogram monitoring system can be worn by said patient throughout normal day-to-day activities without disturbing the patient because there are no long wires between the electrodes and the data acquisition unit.

12. A method as claimed in claim 11, further comprising transmitting said differential signal to a receiver unit.

13. A method as claimed in claim 11, wherein said differential signal is an analog signal.

14. A method as claimed in claim 13, further comprising converting said differential signal to a digital signal.

15. A method as claimed in claim 14, further comprising transmitting said digital signal to a receiver unit.

16. A method as claimed in claim 11, wherein said attaching comprises mounting said data acquisition unit to said first electrode and electrically connecting said data acquisition unit to said second electrode.

17. A method as claimed in claim 11, wherein said data acquisition unit is positioned half way between said first electrode and said second electrode.

18. A method as claimed in claim 12, wherein said transmitter is a RF transmitter.
